(19) United States
(12) Patent Application Publication
(10) Pub. No.: US 2024/0136028 A1
(43) Pub. Date: Apr. 25, 2024

(54) INFORMATION PROCESSING SYSTEM, INFORMATION PROCESSING DEVICE, AND INFORMATION PROCESSING METHOD

(71) Applicants: Preferred Networks, Inc., Tokyo-to (JP); ENEOS Corporation, Tokyo (JP)

(72) Inventors: Kosuke NAKAGO, Tokyo-to (JP); Daisuke TANIWAKI, Tokyo-to (JP); Motoki ABE, Tokyo-to (JP); Marc Alan ONG, Tokyo-to (JP); So TAKAMOTO, Tokyo-to (JP); Takao KUDO, Tokyo (JP); Yusuke ASANO, Tokyo (JP)

(73) Assignees: Preferred Networks, Inc., Tokyo-to (JP); ENEOS Corporation, Tokyo (JP)

(21) Appl. No.: 18/533,469

(22) Filed: Dec. 8, 2023

Related U.S. Application Data
(63) Continuation of application No. PCT/JP2022/023502, filed on Jun. 10, 2022.
(60) Provisional application No. 63/209,420, filed on Jun. 11, 2021.

(51) Int. Cl. G16C 20/70

(52) U.S. Cl. CPC G16C 20/70

(57) ABSTRACT

An information processing system includes a first information processing device and a second information processing device. The first information processing device is configured to receive the atomic information from the second information processing device, calculate a processing result corresponding to the atomic information by inputting the atomic information into a neural network, and transmit the processing result to the second information processing device. The second information processing device is configured to transmit atomic information to the first information processing device.

### CROSS REFERENCE TO THE RELATED APPLICATIONS

This application is continuation application of International Application No. JP2022/023502, filed on Jun. 10, 2022, which claims priority to the U.S. Provisional Patent Application No. 63/209,420, filed on Jun. 11, 2021, the entire contents of which are incorporated herein by reference.

### FIELD

This disclosure relates to an information processing system, an information processing device, and an information processing method.

### BACKGROUND

With the recent development of deep learning, information provision using a model that is based on trained neural networks has come into practice. For example, there are methods of training a neural network model, obtaining NNP (Neural Network Potential) which is an interatomic potential, and performing structure optimization, a molecular dynamics method (Molecular Dynamics), and so on. In these methods, high-load processing of calculating energy from information about a designated atom using the neural network model is sometimes executed a plurality of times based on various conditions, and thus they have a problem of a long time up to the acquisition of a processing result.

### BRIEF DESCRIPTION OF DRAWINGS

FIG. 1 shows a block diagram illustrating an information processing system in an embodiment.

FIG. 2 shows a schematic sequence chart of the whole process in the embodiment.

FIG. 3 shows a block diagram illustrating an example of the hardware configuration in the embodiment.

### DETAILED DESCRIPTION

According to one embodiment, an information processing system includes a first information processing device and a second information processing device. The first information processing device is configured to receive the atomic information from the second information processing device, calculate a processing result corresponding to the atomic information by inputting the atomic information into a neural network, and transmit the processing result to the second information processing device. The second information processing device is configured to transmit atomic information to the first information processing device.

An embodiment of the present invention will be hereinafter described with reference to the drawings. The drawings and the description of the embodiment are presented by way of example and are not intended to limit the present invention.

### Embodiment of the Present Invention

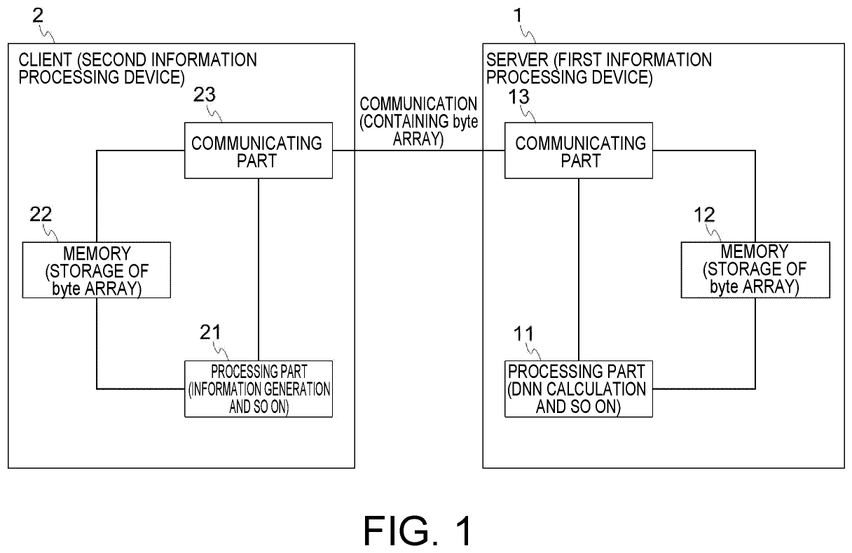

FIG. 1 is a block diagram illustrating an example of an information processing system according to this embodiment. The information processing system according to this embodiment includes a server 1 (an example of a first information processing device) and a client 2 (an example of a second information processing device). The server 1 includes a processing part 11, a memory 12, and a communicating part 13. The client 2 includes a processing part 21, a memory 22, and a communicating part 23. Note that processing performed by the processing part of the server and processing performed by the processing part of the client are different.

The server 1 and the client 2 carry out predetermined processing while communicating with each other. For example, a SaaS (Software as a Service) system in which the server 1 executes software and the client 2 obtains the execution result of the software through a network corresponds to the information processing system according to this embodiment though this is not restrictive.

In SaaS, the high-specification server 1 including GPU (Graphics Processor Unit) and so on executes software with a high processing load and is capable of providing the processing result to the client 2. By using SaaS, even the low-specification client 2 can easily obtain the execution result of the high-load software.

Note that, as a matter of course, the numbers of the servers 1 and the clients 2 are not limited. The information processing system may include a plurality of servers 1, and the servers 1 each may be used by one client 2 or more. Further, in the case where information is transmitted/received between the server 1 and the client 2, one or more devices that relay the communication, such as proxy servers, may be present.

In SaaS, typically, the client 2 transmits, to the server 1, information that the server 1 uses in the execution of the software, and the server 1 transmits information indicating the execution result to the client 2. In the case where an information volume exchanged between the server 1 and the client 2 is large, there is a problem that the time required for the communication (in other words, a required communication time) is long, and it takes a long time for a user of the client 2 to obtain the result of SaaS after giving a SaaS use instruction. Under such circumstances, this embodiment shortens the time required for the processing by the server 1 and the client 2 and their communication by devising an information transmission method.

Processing using a deep neural network model (an example of a model) includes high-load one. For example, there are methods of training a neural network model, obtaining NNP (Neural Network Potential) which is an interatomic potential, and performing structure optimization, a molecular dynamics method (Molecular Dynamics), and so on. These methods execute processing of calculating energy or force using a neural network model for NNP (hereinafter, referred to as an NNP model) from the type and coordinates of each designated atom. In this embodiment, high-load processing such as the calculation of energy or force is executed by the high-specification server 1 including GPU. Data necessary for the processing, for example, the types of used atoms, the positions (coordinates) of the atoms, and so on increase in size according to the number of atoms to be considered. Further, there is also a need for repeating incidental calculations. For example, in some cases, the calculation using the NNP model is executed while the positions of the atoms are repeatedly updated, as in the case where the structure optimization is performed using a BFGS method (Broyden-Fletcher-Glodfarb-Shanno algorithm) or the like and the case where a molecular dynamics simulation is performed based on specific conditions such as temperature and pressure. Further, data transmitted from the server 1 to the client 2, for example, the forces of the respective atoms, increase according to the number of atoms to be considered.

Accordingly, in the case where the function of NNP is provided as SaaS as in this embodiment, communication with a large communication volume is performed a plurality of times between the server 1 and the client 2. Because of this, the communication volume in one communication is preferably as small as possible.

Therefore, in the information processing system of this embodiment, the client 2 transmits information that is to be used in the processing by the server 1, as a byte array usable in this processing to the server 1, and the server 1 uses this byte array at the time of the processing. For example, in the case where calculation using a module of machine learning is executed, the client 2 transmits a byte array of the module to the server 1 without performing data format conversion such as conversion to a data type unique to programming language and serialization according to a transmission method. This eliminates the need for data conversion and serialization by the client 2 and enables a reduction in the processing time in the client 2. Further, since data size typically increases as a result of these conversions, transmitting the byte array without converting it can shorten the communication time. Further, since the server 1 also refers to the received byte array without converting its data format, it is possible to shorten the processing time in the server 1. Further, the server 1 transmits a byte array of information that is based on the processing by the server 1, to the client 2 without performing data format conversion such as conversion to a data type unique to programming language and serialization according to the transmission method. The client 2 refers to the byte array without performing data format conversion.

Hereinafter, the information transmitted by the client 2 to be used in the processing by the server 1 will be referred to as input information. The information based on the processing by the server 1 will be referred to also as output information. The output information may be one indicating the result of the processing or may be one indicating a calculation result in the middle of the processing.

In this embodiment, the case where the server 1 and the client 2 both transmit information as the byte array will be described, but it should be noted that only one of the server 1 and the client 2 may transmit information as the byte array. Further, part of the information transmitted by the server 1 and the client 2 may be transmitted as the byte array.

Further, the case of the transmission as the byte array and the case of the transmission as data that is not a byte array may be switched according to a communication band, communication quality, a time zone when communication is performed, a processing load of each device, and so on. Further, one other than the byte array may be transmitted. For example, it is also possible to transmit the size of the array as a sequence of integers and transmit the pattern of the array as metadata separately from the size of the array.

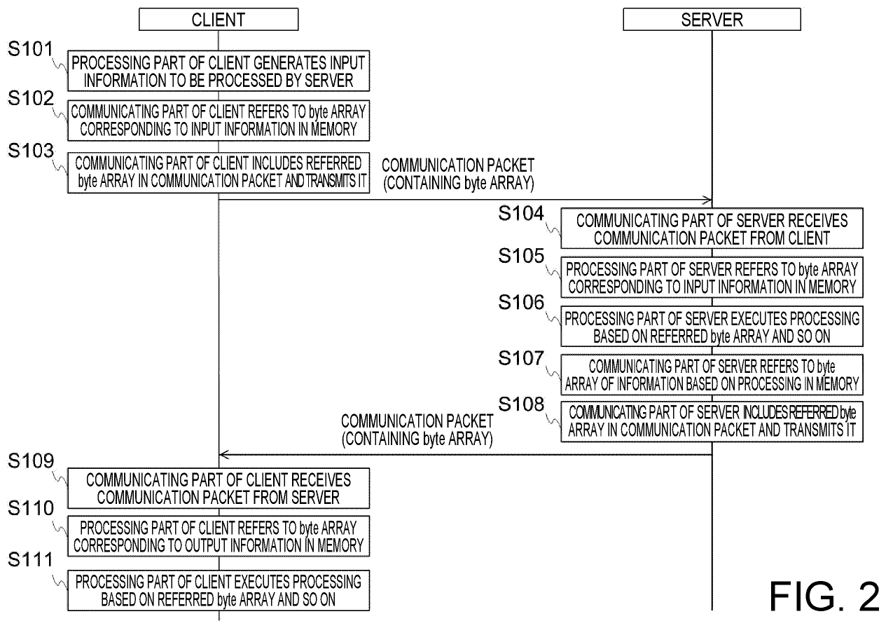

The constituent elements of the server 1 and the client 2 will be described along with the flow of the whole process. FIG. 2 is a schematic sequence chart of the whole process in this embodiment.

The processing part 21 of the client 2 executes designated processes. These processes can be a pre-process of generating information to be used in the processing by the server 1 and a post-process of outputting, to a user, the result of the processing and so on by the server 1. First, the processing part 21 of the client 2 generates the input information to be processed by the server 1 (S101). The input information may be generated according to a predetermined generation method or based on an instruction by the user. For example, in the case where NNP is used, information related to atoms (hereinafter, atomic information) is generated as the input information. The atomic information only needs to include information related to atoms that are to be used in NNP, and includes, for example, information related to the types and positions of the atoms. As the information related to the positions of the atoms, information directly indicating the positions of the atoms by means of the coordinates, information directly or indirectly indicating the relative positions between the atoms, and so on can be cited. Further, the information related to the positions of the atoms may be information representing the positional relationship between the atoms by the distance, angle, dihedral angle, or the like between the atoms. Besides the information on the types and positions of the atoms, the atomic information may include information related to electric charges, information related to the bond of the atoms, information such as periodic boundary condition, cell size, and so on. Further, besides the atomic information, the input information may include information designating a model to be used for NNP, metadata including client and request IDs, and so on. In the case where the atomic information is transmitted as a two- or higher-dimensional array structure (array), it can be conceived to use an array of Numpy which is an extension module for machine learning of programming language Python™, to speed up the processing. The processing part 21 of the client 2 may generate the information in the array format of this Numpy.

Typically, information based on processing by an information processing device is stored as a byte array in a memory of the information processing device. Accordingly, the input information generated by the processing part 21 of the client 2 is stored as a byte array in the memory 22 of the client 2.

The communicating part 23 of the client 2 is in charge of communication with the server 1. The communicating part 23 of the client 2 refers to the byte array corresponding to the input information (atomic information as an example) from the memory 22 (S102). For referring to the byte array, various functions provided in the information processing device may be used. For example, in the case where the information is generated in the array format of the aforesaid Numpy, by executing a predetermined command such as “np.tobytes”, it is possible to refer to the byte array from the memory. Then, the communicating part 23 of the client 2 includes the referred byte array in a communication packet without serializing it and transmits it to the server 1 (S103).

Note that a communication protocol for the exchange of the byte information between the client 2 and the server 1 may be decided as needed. For example, as the communication protocol, gRPC which is one type of RPC (Remote Procedure Call), usable in transport protocol HTTP/2 may be used. Further, description language such as Protocol buffer usable in gRPC may be used. Note that information exchanged between the client 2 and the server 1 may include information not transmitted as a byte array as described above.

The communicating part 13 of the server 1 is in charge of communication with the client 2. The communicating part 13 of the server 1 receives, from the client 2, the communication packet containing the byte array corresponding to the input information (S104). The input information contained in the received communication packet is stored in the memory 12 of the server 1, and since there is no need to deserialize the byte array corresponding to the input information, it is possible to eliminate the processing time which has been required for the deserialization.

The processing part 11 of the server 1 refers to the byte array corresponding to the input information from the memory 12 in order to execute designated processing of SaaS or the like (S105). For referring to the byte array, various functions provided in the information processing device may be used. For example, with a command “np.frombuffer”, it is possible to refer to a byte array corresponding to information in the array format of Numpy, as data that the processing part 11 of the server 1 can handle.

The processing part 11 of the server 1 executes the designated processing of SaaS or the like based on the referred input information and so on (S106). This processing may be performed according to a predetermined method. For example, in the case where the function of NNP is to be provided, the server 1 may input atomic information related to the types, positions, and so on of atoms to a trained NNP model to obtain the processing result such as energy corresponding to the input atomic information from the NNP model. Note that as learning by the NNP model, supervised learning based on ground truth data may be performed. These processing results of the processing part 11 of the server 1 are also stored in the memory 12.

The communicating part 13 of the server 1, similarly to the communicating part 23 of the client 2, refers to the byte array corresponding to information that is based on the processing by the processing part 11 of the server 1 (output information), from the memory 12 (S107). Then, the communicating part 13 of the server 1 includes the referred byte array in a communication packet without serializing it and transmits it to the client 2 (S108). Note that the information based on the processing by the processing part 11 of the server 1 may be not only the processing result (energy as an example) but also a halfway calculation result. For example, it may be an output from an intermediate layer instead of an output from an output layer of the trained neural network model. Further, the information based on the processing by the processing part 11 of the server 1 may also be represented by a two- or higher-dimensional array structure such as an array of Numpy. Note that the server 1 may transmit, to the client 2, a variety of information such as metadata including client and request IDs, besides the information based on the processing by the processing part 11. Note that part of the information transmitted from the server 1 to the client 2 may be transmitted not as the byte array as described above.

For example, the server 1 may improve user convenience by transmitting, to the client 2, information other than the energy which is the result of the forward processing of the NNP model, for example, information such as force and stress which are the results of backward processing.

In the case where the function of NNP is provided as SaaS in this embodiment, the server 1 calculates the processing result (an example of the output information) corresponding to the atomic information received from the client 2 and transmits it to the client 2. The processing result in this embodiment is information calculated based on the atomic information and the NNP model, and may include at least one of energy, information calculated based on the energy, information calculated using the NNP model, and information related to the result of an analysis using an output of the NNP. The information calculated based on the energy may include, for example, information related to at least one of force of each atom, stress (stress of the whole system), virial of each atom, and virial of the whole system. Further, the information calculated using the NNP model may be, for example, charge of each atom. The information related to the result of the analysis using the output of the NNP model may include information obtained after the server 1 additionally analyzes the information calculated using the NNP model. As an example, it may be the result of dynamics calculation (the position of an atom, the speed of an atom, or the like), the calculation result of a physical property value, or the like. The information calculated using the NNP model may be a processing result calculated using the NNP model a plurality of times.

The communicating part 23 of the client 2 receives the communication packet from the server 1 (S109). The output information contained in the received communication packet is stored in the memory 22 of the client 2, and since the byte array corresponding to the output information need not be deserialized, it is possible to eliminate the processing time which has been required for the deserialization.

The processing part 21 of the client 2 refers to the byte array corresponding to the output information from the memory 22 (S110). The referring to the byte array may be done in the same way as is done by the processing part 11 of the server 1. Then, the processing part 21 of the client 2 executes a process based on the referred byte array and so on (S111). For example, the referred byte array is the processing result based on the input information, and the processing part 21 of the client 2 may display this processing result on a monitor or the like so that the user recognizes it.

It is also conceivable that the user recognizing the processing result edits the previous input information and uses SaaS again based on the edited input information. In this case as well, input information is newly generated, and the processes in FIG. 2 are repeated.

As in the above, in this embodiment, the high-load processing using the neural network model is executed by the server 1 capable of higher speed processing than the client 2. In particular, in this embodiment, the high-load processing such as the calculation of energy based on the atomic information is executed by the server 1, whereby the high-speed processing as the whole system is achieved. At this time, information is exchanged as a byte array to speed up the communication between the client 2 and the server 1. Consequently, for example, in such a case where the volume of at least either information input to the neural network model or information output from the neural network model is large, and at least one of updating and downloading of the information exceeds a desired threshold value in normal file communication, the communication time can be made to fall within the desired threshold value.

Note that, in the case where the client 2 has GPU on about the same level as that of the server 1, the time up to the acquisition of the final processing result is shorter when the client 2 executes the calculation using the neural network model, than in this embodiment. However, it is thought that, typically, the number of the clients 2 is larger than the number of the servers 1. Therefore, this embodiment can make the cost lower than that when all the clients 2 that want to execute calculation using the neural network model are equipped with expensive GPUs.

In this embodiment, the plurality of clients 2 may be connected to the server 1. At this time, it is only necessary that the plurality of clients 2 include at least one client that is not capable of executing the processing such as the calculation of energy corresponding to the atomic information using the neural network at a higher speed than the server 1. This also applies to the case where the plurality of clients 2 are connected to the plurality of servers 1.

In this embodiment, the server 1 equipped with the plurality of GPUs intensively processes a plurality of client processes, making it possible to enhance the use efficiency of the GPU resources in the server 1. Further, owing to this, it is possible to lower the processing load in each of the clients 2.

By reading the byte array and transmitting the read byte array to the server as in this embodiment, it is possible to shorten the communication time as compared with the case where it is serialized. Further, by defining the transmission of the byte array in a service definition file or the like, it is possible to transmit the byte array without serializing it. Further, because no overhead is required for file conversion, it is also possible to shorten the processing time in the server and the client.

Note that, in the processing using the NNP model of this embodiment, the atomic information (the types of atoms, the positions of atoms, and so on) transmitted from the client 2 to the server 1 and the processing result (force, charge of each atom, virial of each atom, and so on) transmitted from the server 1 to the client 2 each contain an equal pieces of information to the number of atoms, and thus have a large volume. For example, the atomic coordinates which are one example of the atomic information may have an equal number of values in each of three directions x, y, and z to the number of atoms. Further, force which is one example of the processing result may have an equal number of values of each of three components x, y, and z to the number of atoms. Therefore, by applying the information exchange using the byte array of this embodiment to processing using the NNP model, it is possible to shorten the processing time.

In this embodiment, the calculation of the result of the processing using the NNP model has been mainly described, but the same configuration as this embodiment may be applied to other atomic simulation using the atomic information and the neural network. Further, in this embodiment, the calculation of the processing result using the neural network has been described, but the processing result may be calculated using a model other than the neural network.

Some or all of each device of the server or the client in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage (device) or an auxiliary storage (device)) or outside the computer.

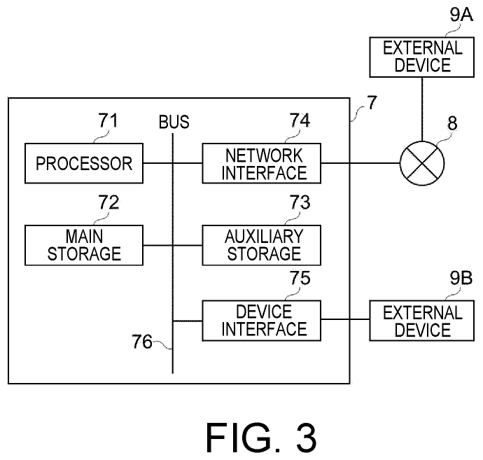

FIG. 3 is a block diagram illustrating an example of a hardware configuration of each device in the above embodiments. As an example, each device may be implemented as a computer 7 provided with a processor 71, a main storage (hereinafter described as a main storage device 72), an auxiliary storage (hereinafter described as an auxiliary storage device 73), a network interface 74, and a device interface 75, which are connected via a bus 76.

The computer 7 of FIG. 3 is provided with each component one by one but may be provided with a plurality of the same components. Although one computer 7 is illustrated in FIG. 3, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device in the above embodiments may be enabled by the main storage device 72 or the auxiliary storage device 73 or may be implemented by a built-in memory built into the processor 71. For example, the memory 12 of the server 1 or the memory 22 of the client 2 in the above embodiments may be implemented in the main storage device 72 or the auxiliary storage device 73.

In the case of each device in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing system comprising:
a first information processing device; and
a second information processing device configured to transmit atomic information to the first information processing device,
wherein the first information processing device is configured to:
receive the atomic information from the second information processing device;
calculate a processing result corresponding to the atomic information by inputting the atomic information into a neural network; and
transmit the processing result to the second information processing device.

2. The information processing system according to claim 1 comprising a plurality of the second information processing devices.

3. The information processing system according to claim 1, wherein the first information processing device is able to execute the calculating the processing result using the neural network at a higher speed than the second information processing device.

4. The information processing system according to claim 1, wherein the neural network is an NNP (Neural Network Potential) model.

5. The information processing system according to claim 1, wherein the second information processing device transmits a byte array of the atomic information stored in a memory of the second information processing device to the first information processing device without converting a data format.

6. The information processing system according to claim 1, wherein the second information processing device transmits a byte array of the atomic information stored in a memory of the second information processing device to the first information processing device without serializing the byte array.

7. The information processing system according to claim 1, wherein the atomic information includes information related to a type of an atom and a position of the atom.

8. The information processing system according to claim 1, wherein the processing result includes at least one of energy, information calculated based on the energy, information calculated using the neural network, or information related to a result of an analysis using an output of the neural network.

9. The information processing system according to claim 1, wherein the first information processing device performs backward processing of the neural network in order to obtain information related to force, and the processing result includes the information related to the force.

10. The information processing system according to claim 9, wherein the first information processing device transmits a byte array of the information related to the force stored in a memory of the first information processing device to the second information processing device without converting a data format.

11. The information processing system according to claim 9, wherein the first information processing device transmits a byte array of the information related to the force to the second information processing device without serializing the byte array.

12. The information processing system according to claim 1, wherein the second information processing device transmits, with the atomic information, at least one of information designating the neural network, metadata about the second information processing device, or metadata about a request for the first information processing device.

13. An information processing method comprising:
receiving, by at least one processor, atomic information from an other information processing device;
calculating, by the at least one processor, a processing result corresponding to the atomic information by inputting the atomic information into a neural network; and
transmitting, by the at least one processor, the processing result to the other information processing device.

14. The information processing method according to claim 13, wherein the at least one processor receives the atomic information from a plurality of the other information processing devices.

15. The information processing method according to claim 13, wherein the at least one processor is able to execute the calculating the processing result using the neural network at a higher speed than the other information processing device.

16. The information processing method according to claim 13, wherein the neural network is an NNP model.

17. The information processing method according to claim 13, wherein the at least one processor refers to the atomic information received from the other information processing device without deserializing the atomic information.

18. The information processing method according to claim 13, wherein the processing result includes at least one of energy, information calculated based on the energy, information calculated using the neural network, or information related to a result of an analysis using an output of the neural network.

19. The information processing method according to claim 13, wherein the calculating the processing result includes performing backward processing of the neural network in order to obtain information related to force, and the processing result includes the information related to the force.

20. The information processing method according to claim 19, wherein the at least one processor transmits a byte array of the information related to the force stored in at least one memory to the other information processing device without converting a data format.

21. The information processing method according to claim 19, wherein the at least one processor transmits a byte array of the information related to the force to the other information processing device without serializing the byte array.

22. An information processing method comprising:
transmitting, by at least one processor, atomic information to an other information processing device; and
receiving, by the at least one processor, a processing result from the other information processing device, the processing result calculated by the other information processing device by inputting the atomic information into a neural network.

23. The information processing method according to claim 22, wherein the other information processing device is able to execute the calculating the processing result using the neural network at a higher speed than the at least one processor.

24. The information processing method according to claim 22, wherein the neural network is an NNP model.

25. The information processing method according to claim 22, wherein the at least one processor transmits a byte array of the atomic information stored in at least one memory to the other information processing device without converting a data format.

26. The information processing method according to claim 22, wherein the at least one processor transmits the byte array of the atomic information to the other information processing device without serializing the byte array.

27. The information processing method according to claim 22, wherein the atomic information includes information related to a type of an atom and a position of the atom.

28. The information processing method according to claim 22, wherein the processing result includes information related to force obtained by the other processing device performing backward processing of the neural network.

29. An information processing device comprising:
at least one memory; and
at least one processor configured to:
receive atomic information from an other information processing device;
calculate a processing result corresponding to the atomic information by inputting the atomic information into a neural network; and
transmit the processing result to the other information processing device.

30. The information processing device according to claim 29, wherein the at least one processor performs backward processing of the neural network in order to obtain information related to force, and the processing result includes the information related to the force.

31. An information processing device comprising:
at least one memory; and
at least one processor configured to:
transmit atomic information to an other information processing device; and
receive a processing result that the other information processing device calculates by inputting the atomic information into a neural network, from the other information processing device.

32. The information processing device according to claim 31, wherein the processing result includes information related to force obtained by the other processing device performing backward processing of the neural network.
